(19) United States
(12) Patent Application Publication
(10) Pub. No.: US 2010/0234653 A1
(43) Pub. Date: Sep. 16, 2010

(54) PROCESSES FOR MAKING ALKYL HALIDES

(75) Inventors: Mitchel Cohn, West Haven, CT (US); Steven J. Hobbs, Wolcott, CT (US)

Correspondence Address: Patent Administrator;Chemtura Corporation, 199 Benson Road, Middlebury, CT, 06749, US

(73) Assignee: Chemtura Corporation, Middlebury, CT (US)

(21) Appl. No.: 12/403,106

(22) Filed: Mar. 12, 2009

(51) Int. Cl. C07C 17/16; B01J 14/00; B01J 19/00

(52) U.S. Cl. 570/258; 422/198; 422/189

(57) ABSTRACT

The invention is directed to processes for producing an alkyl halide, preferably isobutyl bromide. In one embodiment, the process comprises the steps of: (a) contacting an alcohol with a hydrogen halide in a reactor at elevated temperature under conditions effective to form an initial product mixture comprising the alkyl halide, the alcohol, the hydrogen halide and water; (b) cooling the initial product mixture to form a cooled organic phase positioned above a cooled aqueous phase; (c) separating the cooled organic phase from the cooled aqueous phase. The process preferably further comprises a step of: (d) heating at least a portion of the cooled aqueous phase under conditions effective to form additional alkyl halide.

### FIELD OF THE INVENTION

The present invention relates to the production of alkyl halides. More specifically the invention relates to producing alkyl halides such as isobutyl bromide by utilizing anhydrous hydrogen bromide and two reaction stages.

### BACKGROUND OF THE INVENTION

Isobutyl bromide (IBB or 1-bromo-2-methylpropane) is a product having considerable industrial importance, particularly in the synthesis of various organic compounds. In principle, IBB is the product of a reaction between hydrobromic acid (HBr) and isobutanol (IBA). Depending on how the reaction is carried out, however, the result may be unsatisfactory as to selectivity and/or purity of the product or contamination with by-products, particularly tert-butyl bromide (TBB).

The bromination reaction is well-known and may be represented by the following reaction:

R—OH+HBr → R—Br+H₂O

Generally, equilibrium between reactants and products is attained, as described by Migrdichian, “Organic Synthesis,” Vol. 1, p. 17, 1957, Reinhold Publishing Corp. In practice, the preparation of alkyl halides, e.g., IBB, in aqueous solution of HBr has been impractically slow, inefficient and in some cases even impossible to achieve. For this reason, the art has not provided an efficient process for producing alkyl-halides, in particular for producing IBB, in aqueous solutions of HBr with good yield and industrially acceptable rates.

HBr, most lower alcohols such as IBA, some byproducts formed in the bromination reaction and IBB form low boiling azeotropes with water. In addition, IBB forms an azeotrope with IBA. As a result, it is impractical to distill the product alkyl halide (e.g., IBB) and water, as formed, and hope to maintain a constant HBr to alcohol ratio in the reaction mixture.

To promote such reactions, it is common practice either to employ anhydrous HBr and operate in the vapor phase and/or to employ dehydrating agents. Sulfuric acid, for example, is commonly used for this purpose, sometimes both as a means of generating HBr from a convenient salt (thus operating in an almost anhydrous mode) as well as to serve as a condensation agent.

As an alternative approach, alkyl bromides, e.g., IBB, can be produced by reacting an alcohol with elemental bromine and either phosphorus or sulfur. Elemental bromine, however, is more difficult to obtain than HBr which is obtainable as a by-product in bromination reactions. Furthermore, the costs associated with using phosphorus or sulfur are exacerbated since these elements are converted into the corresponding acids in the reaction and may become ecologically troublesome wastes.

Additional processes for producing alkyl halides are widely known in the art. U.S. Pat. No. 7,217,849 to Meirom et al., for example, discloses a method for producing n-propyl bromide of a high degree of purity, which contains isopropyl bromide in an amount lower than 0.1% w/w, and usually lower than 0.05%. The method is characterized in that n-propanol is reacted with HBr which is in gas form, preferably dry, and which is in excess over the stoichiometric amount, wherein the excess HBr is at the end of the reaction in an aqueous solution formed from the reaction water. The invention further relates to N-propyl bromide of high purity, containing typically less than 500 ppm of isopropyl bromide.

As another example, U.S. Pat. No. 5,138,110 to Segall et al. discloses a process for preparing lower alkyl-halides from the reaction of HX and the corresponding lower alcohol, wherein X represents a halogen atom. The process comprises continuously feeding HX and a lower alcohol to a reactor maintaining the instantaneous molar ratio of HX to the alcohol greater than 3, and continuously distilling off lower alkyl-halide and water from the reactor, continuously separating the lower alkyl-halide and water and recycling part of the said water to the distillation column to abate HX distillation from the reactor. High acid concentration and temperatures are maintained to obtain high yields and rapid conversion of alkanols.

U.S. Pat. No. 5,384,415 to Mas et al. discloses a process for the preparation of a brominated compound which comprises the step of reacting at least one compound selected from the group consisting of a chloroformate, a chlorosulfite and a chlorophosphite with a brominating agent for a time sufficient to obtain at least one brominated compound. In particular, an alcohol is converted into a chloroformate, chlorosulfite or a chlorophosphite, which is then brominated to obtain the desired product. In another embodiment, a brominating agent is reacted with a reactant selected from the group consisting of thionyl chloride, phosgene and phosphorous oxychloride, followed by contacting the reaction product obtained with an alcohol to be brominated.

Even in view of these known processes for producing alkyl halides, the need remains for cost-effective processes for forming IBB at high conversions and selectivity. In addition, the high cost of materials of construction and the extremely corrosive nature of the HBr employed in the bromination reaction drives the need for processes employing minimal equipment.

### SUMMARY OF THE INVENTION

The present invention is directed to processes for producing alkyl halides, and particularly isobutyl bromide. In one embodiment, the invention is to a process for producing an alkyl halide, comprising: (a) contacting an alcohol with a hydrogen halide in a reactor at elevated temperature, e.g., 70 to 80° C., under conditions effective to form an initial product mixture comprising the alkyl halide, the alcohol, the hydrogen halide and water; (b) cooling the initial product mixture to form a cooled organic phase positioned above a cooled aqueous phase; and (c) separating the cooled organic phase from the cooled aqueous phase. In one aspect, the process further comprises: (d) heating, preferably reactively distilling, at least a portion of the cooled aqueous phase under conditions effective to form additional alkyl halide. Thus, in this aspect, the alkyl halide product is formed in two reaction stages. Preferably, the alcohol is isobutanol, the alkyl halide is isobutyl bromide, and the hydrogen halide is hydrogen bromide. The processes desirably provide the ability to form very pure, e.g., greater than 95 wt. % pure, final alkyl halide products without the need for a separate distillation step.

In preferred embodiments, anhydrous hydrogen bromide is added to the reactor during the contacting step. The reactor optionally includes a circulation loop in which at least a portion of the contacting occurs, and a circulation stream comprising the isobutanol, the hydrogen bromide, the isobutyl bromide and the water is pumped through the circulation loop. Thus, the process optionally further comprises the step of (e) pumping a circulation stream comprising the isobutanol, the hydrogen bromide, the isobutyl bromide and the water to the reactor in a circulation loop. In this aspect, the gaseous anhydrous hydrogen bromide optionally is added to the circulation stream. Optionally, catalyst is added during the heating step, e.g., the reactive distillation step. Additional hydrogen bromide may be added during the optional reactive distilling step.

Preferably, the alkyl halide, e.g., isobutyl bromide, formed in the processes of the invention are post-processed to form a final alkyl halide product. The alkyl halides formed in the two reaction stages may be post-processed separately or collectively. The process preferably comprises washing the alkyl halide, e.g., isobutyl bromide, with water and/or base to form washed alkyl halide. The process also preferably comprises a step of drying the washed alkyl halide, e.g., with a molecular sieve material, to form a final alkyl halide product.

As indicated above, the optional second reaction stage preferably comprises reactive distillation, which preferably produces an overhead stream comprising at least a portion of the additional isobutyl bromide. The overhead stream optionally is cooled and condensed to form a liquid product stream. The resulting additional isobutyl bromide may be combined with the isobutyl bromide in the cooled organic phase to form a combined crude product stream which may be post processed, as described above.

The ultimately formed final alkyl halide product, e.g., final isobutyl bromide product, preferably comprises at least 95 weight percent alkyl halide, e.g., isobutyl bromide, based on the total weight of the final alkyl halide product.

In another embodiment, the invention is to a process for producing an isobutyl halide, e.g., isobutyl bromide, comprising: (a) reacting isobutanol with a hydrogen halide, e.g., hydrogen bromide, to form an aqueous phase situated on top of an organic phase; (b) cooling the aqueous phase and the organic phase such that the phases invert; and (c) separating the organic phase from the aqueous phase. The process preferably further comprises a step of: (d) heating the aqueous phase to form additional isobutyl bromide.

In another embodiment, the invention is to a reaction system for forming an alkyl halide, comprising: (a) a reactor for contacting a hydrogen halide with an alcohol under conditions effective to form an organic phase comprising the alkyl halide and an aqueous phase comprising water, the hydrogen halide and the alcohol; and (b) means for cooling and separating the organic phase and aqueous phase to form a cooled organic phase and a cooled aqueous phase. The system preferably further comprises (c) a reactive distillation unit in fluid communication with the reactor for receiving at least a portion of the cooled aqueous phase and configured to form additional alkyl halide from the hydrogen halide and the alcohol contained in the cooled aqueous phase. Optionally, the cooled organic phase is directed to a washing vessel, which optionally is the reactor, and an overhead stream from the reactive distillation unit directs the additional alkyl halide to the washing vessel. As above, ideally, the alcohol is isobutanol, the alkyl halide is isobutyl bromide, and the hydrogen halide is hydrogen bromide. The wash vessel preferably is in fluid communication with a drying unit. The reaction system may be a batch system or a semi-continuous reaction system.

In another embodiment, the invention is directed to a continuous reaction system for forming an alkyl halide, comprising: (a) a reactor for contacting a hydrogen halide with an alcohol under conditions effective to form a product mixture comprising the alkyl halide, water, the hydrogen halide and the alcohol; and (b) a reactive distillation unit in fluid communication with the reactor for receiving at least a portion of the product mixture and configured to form a crude alkyl halide product mixture comprising alkyl halide from the reactor and additional alkyl halide formed from the hydrogen halide and the alcohol contained in the product mixture. As above, preferably the alcohol is isobutanol, the alkyl halide is isobutyl bromide, and the hydrogen halide is hydrogen bromide. The reactive distillation unit optionally is in fluid communication with a washing unit, and the crude alkyl halide product mixture is washed in the washing vessel to form a washed stream. The washing unit optionally is in fluid communication with a phase separation unit for separating an organic phase comprising the alkyl halide, and an aqueous phase. The phase separation unit optionally is in fluid communication with a drying unit configured to dry the organic phase to form a final alkyl halide product.

### BRIEF DESCRIPTION OF THE DRAWINGS

The present invention will be better understood in view of the non-limiting figures, wherein like characters refer to the same or similar parts throughout the views, and in which:

FIG. 1 is a schematic diagram of a process for producing isobutyl bromide in accordance with an embodiment of the present invention;

FIG. 2A-2C are schematic diagrams showing a phase inversion occurring in the process of FIG. 1;

FIG. 3A-3B are cross-sections of exemplary devices for introducing hydrogen bromide into a pumparound stream; and

FIG. 4 is a schematic diagram of a process for producing isobutyl bromide in accordance with an embodiment of the present invention.

### DETAILED DESCRIPTION OF THE PREFERRED EMBODIMENTS

The present invention relates to the production of alkyl halides, and in particular to the production of isobutyl bromide (IBB), which, for example, may be utilized in the synthesis of various organic compounds. Typically, the production of alkyl halides involves the reaction of alcohols with an acid of the respective halide. Generally, this reaction takes place inefficiently and does not achieve a high rate of selectivity of the desired product, e.g., IBB. In contrast, the processes of the present invention, in which the resulting crude reaction mixture is cooled prior to phase separation, typically provide improved selectivity. In preferred embodiments, the process forms the alkyl halide (e.g., IBB) in two reaction stages further maximizing selectivity and yield. In this manner, selectivities greater than 90%, greater than 95%, greater than 98%, greater than 99% or greater than 99.5%, of the desired product, preferably IBB, when compared to other reaction products, e.g., tert-butyl bromide (TBB) may be achieved. In addition, the processes of the present invention provide high conversions and yields without the need for separate distillation steps and concomitant expense in order to isolate the IBB.

As used herein, the term “conversion” means the amount of reactant, e.g., isobutanol (IBA) and hydrogen bromide (HBr), that is converted to product, e.g., IBB and water. The term “selectivity” means the amount of reactants that are converted into a specified product, e.g., IBB. “Yield” refers to the product of conversion and selectivity for the specified product.

In one embodiment of the present invention, an alcohol, e.g., IBA is reacted with a hydrogen halide, e.g., HBr to form the equivalent alkyl halide (e.g., alkyl bromide such as IBB) and water, as shown below:

R—OH+HX → R—X+H₂O

wherein R represents an alkyl group and X represents a halogen atom.

Although the present invention is described with reference to the formation of isobutyl bromide from isobutanol and hydrogen bromide, it should be understood that the invention is not so limited and may be used to form a variety of alkyl halides from a variety of possible starting alcohols. For example, the process may be used to form one or more of the following alkyl bromides (or other halides) from the corresponding alcohol: methyl bromide, ethyl bromide, n-propyl bromide, and n-butyl bromide. Similarly, although discussed herein in terms of hydrogen bromide, the hydrogen halide employed in the processes of the invention may be selected from hydrogen fluoride, hydrogen chloride, hydrogen bromide, hydrogen iodide and hydrogen astatide.

The reaction of IBA and HBr to produce IBB and water has the potential to produce a minor amount of byproducts such as n-butyl bromide (NBB), sec-butyl bromide (SBB) and TBB. Typically, it is desirable to maximize the selectivity of IBA and HBr to IBB and minimize selectivity to NBB and TBB (as well as other by-products) in the product mixture.

HBr is a diatomic molecule, which, under standard temperature and pressure, is a gas. HBr can be liquified to form an aqueous solution of hydrobromic acid by dissolving HBr in water. Conversely, HBr can be liberated from hydrobromic acid solutions upon the addition of a dehydration agent such as sulfuric acid.

In a preferred embodiment, anhydrous HBr is utilized in the bromination reaction. By utilizing anhydrous HBr, water content in the reaction mixture is advantageously minimized, thereby maximizing HBr reactivity with IBA to form IBB and water. By utilizing anhydrous HBr in accordance with the invention, a high selectivity of IBB is surprisingly and unexpectedly achieved. The use of anhydrous HBr also facilitates the production of IBB without necessitating a separate distillation step, thereby simplifying the process, increasing overall efficiency and reducing cost. Of course, in some aspects of the invention, one or more distillation steps may be employed to further purify the IBB product and/or recover residual IBB from the aqueous phase.

In addition, the use of anhydrous HBr in accordance with the present invention results in high conversion. In a preferred embodiment, the conversion is at least about 70%, e.g., at least about 80%, at least about 90%, at least about 95% or at least about 99%. The processes of the invention preferably form product mixtures that are rich in IBB as formed, e.g., without product distillation. For example, the combined product of the first and second reaction stages may comprise at least 93 wt. %, e.g., at least 96 wt. % or at least 98.5 wt. % IBB as formed (without a separate distillation step).

The source of the anhydrous HBr may vary widely. In one embodiment, the anhydrous HBr is produced by reacting sodium bromide (NaBr) with a non-oxidizing acid, e.g., phosphoric acid or acetic acid. Alternatively, anhydrous HBr may be produced by the bromination of tetraline or by the reaction of purified hydrogen gas and bromine in the presence of a catalyst, e.g., platinum catalyst.

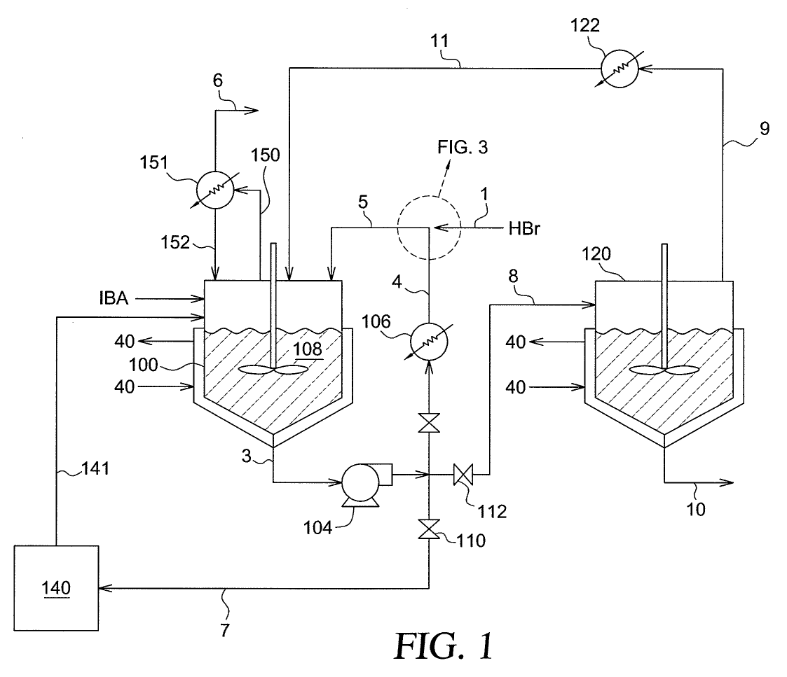

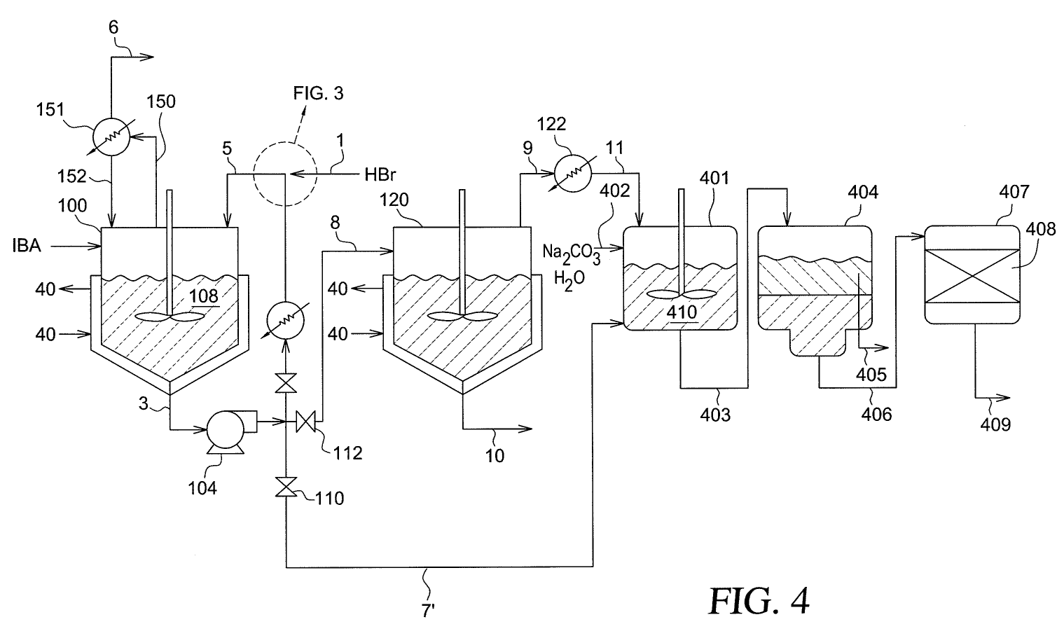

Preferably, the anhydrous HBr contains water in an amount less than 0.1 wt %, e.g., less than 0.01 wt. %, or less than 0.001 wt. %, based on the total weight of the anhydrous HBr. The anhydrous HBr should comprise HBr in an amount greater than 98 wt. %, preferably greater than 99 wt. %, but may further comprise elemental hydrogen and/or bromine in very minor amounts, e.g., in an amount less than 1 wt. %, e.g., less than 0.5 wt. % or less than 0.1 wt. %, separately or in combination. Preferably, the anhydrous HBr is added to the reaction mixture in gas form through a conduit in a recirculation loop, as described in greater detail below with reference to FIGS. 1 and 3.

The IBA composition that is employed in the process of the invention may or may not be substantially pure. In some embodiments, for example, the IBA composition comprises IBA in an amount no less than 95 wt. %, no less than 99.0 wt. %, no less than 99.8 wt. %, or no less than 99.95 weight percent, based on the weight of the IBA composition. In terms of ranges, the IBA composition may comprise from 95 to 99.99 wt. % IBA, e.g., from 99.8 to 99.99 wt. % or from 99.8 to 99.95 wt. % IBA. The IBA may have some acidity due to the presence of a minor amount of acetic acid, which may be present in an amount no greater than 0.005 wt. %, based on the total weight of the IBA composition. The IBA preferably has an APHA color value, as determined by ASTM D 1209, of no greater than 10, e.g., no greater than 9 or no greater than 8. Also, like the anhydrous HBr, the IBA composition preferably is substantially dry and, for example, preferably contains no more than 0.1 wt. % water, no more than 0.05 wt. % water, or no more than 0.01 wt. % water, based on the weight of the IBA composition.

### First Reaction Stage and Phase Inversion

As shown in FIG. 1, in a preferred embodiment, HBr, e.g., anhydrous HBr, contacts IBA in reactor 100 in a first reaction stage to form IBB and water. In a preferred embodiment, the IBA is charged to reactor 100 before the introduction of the HBr, and the HBr is then added, preferably as a gas, to reactor 100 to contact the IBA. As shown, the anhydrous HBr comes into contact with at least a portion of the IBA outside of reactor 100, e.g., before entering reactor 100. For example, HBr, e.g., anhydrous HBr, may be added to a circulation loop that recycles the reaction mixture to reactor 100, optionally with cooling to control reactor temperature. In FIG. 1, the circulation loop comprises streams 3, 4 and 5. In another embodiment, the HBr, e.g., anhydrous HBr, contacts and reacts with a portion of the IBA outside of reactor 100 and contacts and reacts with the remainder of the IBA within reactor 100. In other embodiments, the IBA is added directly to the reactor without first contacting IBA externally to the reactor. For example, the HBr may be bubbled into the reactor below the surface of the reaction mixture.

In a preferred embodiment, the reaction takes place at an elevated temperature. The reaction may take place, for example, at a temperature greater than 30° C., e.g., greater than 50° C., greater than 70° C., greater than 100° C., or greater than 200° C. In terms of ranges, the reaction may take place at a temperature ranging from 30° C. to 400° C., e.g., from 40° C. to 300° C., from 40° C. to 200° C., from 50° C. to 100° C. or from 70° C. to 80° C. Due to the exothermic nature of the reaction, it may be desirable to cool the reactor, optionally with a heat exchanger 106 contained in a circulation loop. The contacting of the HBr and IBA preferably takes place substantially at ambient pressure, although in some embodiments, the reaction may take place at elevated pressure.

In a preferred embodiment, the IBA and the HBr contact one another with mixing, e.g., the IBA and HBr are continuously mixed with one another in reactor 100. For example, the IBA and the HBr may be mixed with a mixer, e.g., an impeller-type mixer 102, to form an initial reaction mixture, e.g., initial product mixture 108, shown in FIG. 1. Additional mixing devices that may be employed include, for example, sonic mixers, high shear mixers, stir bars, and the like.

In one embodiment of the invention, a catalyst is utilized to further promote the reaction. The catalyst may, for example, be added to the reaction mixture during the contacting step. Additionally or alternatively, the catalyst may be added to the reactor and/or to the HBr and/or the IBA prior to introduction to the reactor. As a further alternative, the catalyst may be added to the HBr and/or the IBA via the circulation loop. Catalysts for such applications include, for example, activated carbon, granulated active carbon, impregnated active charcoals, phosphorous-containing catalysts, aluminas, impregnated aluminas, and metal compound catalysts.

In a preferred embodiment, the reaction takes place in a stoichiometric excess of HBr. The excess of HBr drives the reaction toward completion, maximizes conversion of the IBA and aids separation of crude reaction product. As indicated above, water is formed as a byproduct of the bromination reaction. As a result, after mixing is stopped, unreacted (excess) HBr will separate into an aqueous phase. Providing excess HBr increases the weight of the resulting aqueous phase, thereby enhancing separation of the crude reaction product. In a preferred embodiment, the HBr and the IBA are contacted, e.g., added to the reactor, at an initial molar ratio greater than 1:1, e.g., greater than 1.2:1, greater than 1.4:1 or greater than 1.6:1. In terms of ranges, the HBr and IBA are added to the reactor in a molar ratio of from 1.2 to 3.0, e.g., from 1.4 to 2.0 or from 1.4 to 1.8.

In a preferred embodiment, the progress of the reaction is monitored by measuring the density of the reaction mixture. The measurement of the reaction mixture, for example, may be performed intermittently and/or continuously. The alkyl halide product, e.g., IBB, will have a density that is greater than the reactant alcohol. As a result, as the reaction proceeds and as IBA is converted to IBB, the density of the reaction mixture will increase. Accordingly, when the density of the reaction mixture achieves a predetermined level or when the density remains substantially constant, e.g., when the density plateaus, the reaction may be considered substantially complete. By monitoring the density of the product stream in such a manner, maximum conversion can be achieved.

Additionally or alternatively, the progress of the reaction may be monitored by measuring the amount of gaseous HBr that exits the reactor. In this embodiment, a stream of gaseous anhydrous HBr 1 is fed to reactor 100, as shown in FIG. 1 or, alternatively, through a sub-surface HBr feed source. At the beginning of the reaction, the HBr reacts with the IBA to produce IBB and water. As the reaction proceeds toward completion, the IBA in the reaction mixture is consumed. Thus, when the IBA is substantially depleted from the reaction mixture, excess gaseous HBr that is added to the reactor does not react with IBA, exits the reactor via stream 150 and is directed to condenser 151 to form vent stream 6 and condensed stream 152. Accordingly, when vent stream 6 comprises HBr in an amount substantially the same as the HBr being added to the reaction system, the reaction may be considered substantially complete. In this manner, reaction progress may be determined by monitoring HBr flow rate in the feed gas as compared to the HBr flow rate in vent stream 6.

In one embodiment, the vent stream is further processed for disposal or is recycled. Separation or purification devices that are known in the art can be used to process vent stream 6. In a preferred embodiment, vent stream 6 is directed to a scrubber (not shown). As vent stream 6 is scrubbed, the scrubber temperature increases due to the heat of solution and/or heat of reaction of neutralization. Accordingly, scrubber temperature generally corresponds to HBr flow rate in vent stream 6. As such, the scrubber temperature may be utilized to monitor the progress of the reaction.

In another embodiment, gas chromatography/mass spectroscopy (GC/MS) is utilized to detect the components and concentrations thereof that are present in the product stream. For example, the progress of the reaction may be monitored by observing the peak associated with IBA. As the peak disappears, the reaction may be considered substantially complete.

At some point, after a desired amount of the IBA has reacted, the mixing is terminated, preferably after a high conversion has been achieved, e.g., a conversion of at least about 70%. Upon substantial completion of the reaction, the flow of HBr to the reactor is stopped.

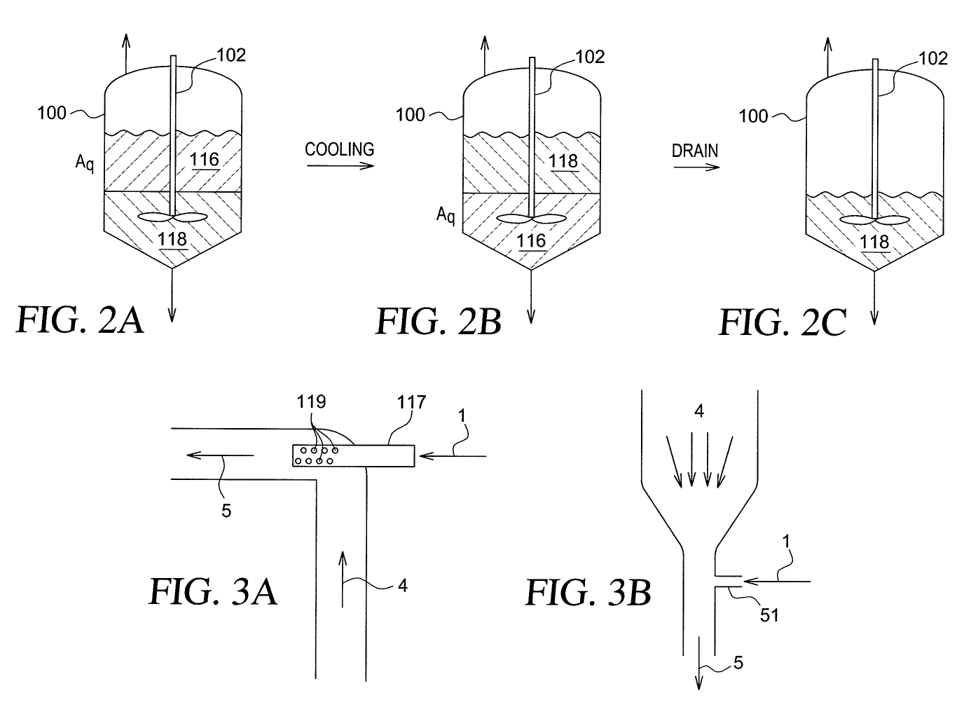

As shown in FIG. 2A, in a preferred embodiment, upon termination of the reaction in accordance with one embodiment of the invention, the resultant initial product mixture comprises aqueous phase 116 and organic phase 118. In the initial product mixture, aqueous phase 116 is positioned above organic phase 118 in reactor 100. Generally, the aqueous phase comprises primarily water and HBr, and may further comprise minor amounts of IBA, IBB, TBB, SBB and NBB. The organic phase generally comprises primarily IBB and TBB and may further comprise minor amounts of water, HBr, IBA, SBB, NBB and other reaction by-products.

In a preferred embodiment of the invention, the initial product mixture is cooled to form a cooled product mixture. As the initial product mixture is cooled, the relative densities of the organic phase and the aqueous phase surprisingly and unexpectedly invert with respect to one another. That is, the density of the aqueous phase becomes greater than the density of the organic phase. Accordingly, by cooling the initial product mixture in accordance with the invention, the aqueous phase and the organic phase are spatially inverted, as shown in the transition from FIG. 2A to FIG. 2B, which illustrates cooled organic phase 118 situated above cooled aqueous phase 116. By inverting, the two phases pass through one another and advantageously provide an in situ extraction step further isolating IBB from any excess reactants and byproduct water. In various optional embodiments, initial product mixture 108 is cooled to less than 50° C., e.g., less than 40° C. or less than 30° C. in order to form cooled product mixture. In terms of ranges, the initial product mixture may be cooled to a temperature from −10° C. to 50° C., e.g., from 10° C. to 40° C. or from 20° C. to 30° C. Typically, the phase inversion is observed at between about 50° C. and about 75° C.

The cooling may be achieved, for example, by applying an ice bath to the initial product mixture. In another embodiment, the cooling is achieved by applying a cooling jacket or a cooling coil to the reactor. For example, cooling may be achieved by circulating a cooling agent, such as cooled water, around reactor 100. In another embodiment, the cooling is achieved by turning off or removing the heating element and allowing the initial product mixture to cool to room temperature. In another aspect, the cooling is achieved with heat exchanger 106 in a circulation loop (streams 3, 4 and 5). Of course, other suitable cooling methods known in the art can be utilized to cool the initial product mixture.

In a preferred embodiment of the invention, the IBB concentration in the cooled organic phase is greater than the IBB concentration in the initial organic phase. The IBB concentration (weight percent) in the cooled organic phase, for example, may be greater than the IBB concentration in the initial organic phase, for example, by at least 3%, e.g., at least 5% or at least 10%. Conversely, the IBB concentration (weight percent) in the cooled aqueous phase preferably is less than the IBB concentration in the initial aqueous phase, for example, by at least 3%, at least 5% or at least 10%.

The IBA in the initial product mixture ideally follows the aqueous HBr phase during the phase inversion such that substantially all of the IBA remains in the aqueous phase throughout the cooling step. Without being bound by theory, residual IBA originally contained in the organic phase desirably may be separated into the cooled aqueous phase through the inversion and cooling step, as shown in Example 3, below.

In a particularly preferred embodiment, the reactor is continuously mixed, e.g., with an agitator, during the cooling step. As a result, in this embodiment, initial organic and aqueous phases, i.e., at elevated temperatures, are not formed. Once the reaction mixture has cooled to a desired temperature, e.g., a temperature less than 40° C., less than 30° C., less than 25° C. or less than 20° C., the mixing is stopped and the phases are allowed to separate to form the cooled aqueous phase and the cooled organic phase.

Although the specific compositions of the cooled organic phase and the cooled aqueous phase may vary widely depending on the reaction conditions employed, the cooled organic phase preferably comprises IBB in an amount greater than about 90 weight percent, greater than about 95 weight percent or greater than 97 weight percent, based on the weight of the cooled organic phase. In some exemplary embodiments, the cooled aqueous and organic phases may comprise water, HBr, IBA, IBB, SBB, NBB and TBB in the ranges provided below in Table 1.

TABLE 1
Approximate Compositions of Cooled Aqueous and Organic Phases

| | Cooled Aqueous | | | Cooled Organic | | |
|---|---|---|---|---|---|---|
| Com- | Phase (Wt. %) | | | Phase (Wt. %) | | |
| ponent | Broad | Narrow | Narrower | Broad | Narrow | Narrower |
| Water | 40-90 | 40-80 | 50-70 | <5 | <3 | <1 |
| HBr | 10-60 | 20-60 | 40-50 | <5 | <3 | <1 |
| IBA | 1-30 | 1-25 | 1-20 | <5 | <3 | <1 |
| IBB | <5 | <3 | <1 | 75-99 | 85-99 | 95-99 |
| SBB | <1 | <0.5 | <0.3 | <1 | <0.5 | <0.3 |
| TBB | <1 | <0.5 | <0.3 | 0.1-10 | 1-8 | 1-5 |
| NBB | <1 | <0.5 | <0.3 | <1 | <0.5 | <0.3 |
| Other | <1 | <0.5 | <0.3 | <1 | <0.5 | <0.3 |

As shown in FIGS. 2B and 2C, after phase inversion, the cooled aqueous phase 116 (the lower phase) is removed from the reactor and stored in a first vessel, and the cooled organic phase 118 comprising IBB (the upper phase) is removed from the reactor and directed to a second vessel. Reverting to FIG. 1, in one embodiment, valve 112 is opened, valve 110 is closed and cooled aqueous phase is drained out of reactor 100 to form cooled aqueous stream 8. Cooled aqueous stream 8 may be further processed, as discussed below. When the cooled aqueous stream 8 has been completely evacuated from reactor 100, valve 112 is closed. At this point, the remaining product mixture contained in reactor 100 comprises cooled organic phase 118, as shown in FIG. 2C and FIG. 1. Valve 110 is opened, and the remaining product mixture, e.g., the cooled organic phase 118, which comprises the product IBB, is drained out of reactor 100 forming cooled organic stream 7, which is directed to IBB storage vessel 140. Vessel 140 holds and/or stores the IBB from cooled organic stream 7. The use of valve 100, valve 112 and vessel 140 are merely exemplary devices that can be used in the practice of the invention. Alternative flow control devices and collection schemes may, of course, be utilized to drain, separate and/or store the aqueous and organic phases created in the above-mentioned reaction system. In a preferred embodiment, the cooled organic phase 118 is washed, e.g., with water and/or a dilute soda ash solution, and dried, e.g., with molecular sieves, in reactor 100 and/or in vessel 140.

As indicated above, in preferred embodiments, the process includes utilizing a circulation loop to circulate the reaction mixture, which comprises IBA, HBr, IBB and water, to reactor 100. In a preferred embodiment, the circulation loop includes stream 3, pump 104, heat exchanger 106, cooled stream 4 and feed stream 5, all shown in FIG. 1. Stream 3 comprises IBA, HBr, IBB, water and possibly a minor amount of SBB. Pump 104 is utilized to pump stream 3 out of the reactor and through the circulation loop. Because of the exothermic nature of the reaction, stream 3 exits the reactor at an elevated temperature, e.g., at a temperature greater than 75° C. or at a temperature greater than 90° C. In a preferred embodiment, it is desirable to cool stream 3 to control reaction temperature, as discussed above. As shown in FIG. 1, heat exchanger 106 cools stream 3, for example, by at least 20° C., or by at least 50° C. A cross-current heat exchanger or a shell and tube heat exchanger, for example, may be used to cool stream 3. Of course, any suitable heat exchanger or cooler or combination thereof may also be utilized.

In a preferred embodiment, gaseous anhydrous HBr stream 1 is added to stream 4 to form feed stream 5, which is introduced to reactor 100. This is shown in FIG. 1, 3A and 3B. One mechanism that may be employed to introduce HBr, e.g., anhydrous HBr, into stream 4 is shown in FIG. 3A. As shown, inlet conduit 117 conveys HBr stream 1 into cooled stream 4 thereby combining the two streams 1, 4 to form feed stream 5. Inlet conduit 117 preferably includes a nozzle having a plurality of exit holes 119 therein, which facilitate mixing between the introduced HBr and the IBA (and other components) contained in stream 4. In another embodiment, a solid conduit with a single exit hole may be utilized.

In another embodiment, shown in FIG. 3B, as pump 104 drives the circulation loop, stream 4 flows over and past an inlet conduit 51. In doing so, a Venturi effect is created forming a pressure drop across inlet conduit 51, which draws the HBr, e.g., anhydrous HBr, from its source via inlet conduit 51. In so doing, HBr back-flow into inlet conduit is inhibited. Resulting feed stream 5 is directed to reactor 100. By introducing the HBr into feed stream 5 and eventually into reactor 100 in this manner, the addition of the HBr is more effectively and safely controlled.

In an alternative embodiment, anhydrous HBr may be fed to the reactor volume directly from the HBr source. In such a configuration, the HBr/IBA reaction takes place exclusively within the reactor. In one embodiment, the HBr may be bubbled through the IBA in the reactor.

In a preferred embodiment, the process is performed in a batch or semi-batch process. In such a process, the reaction is ultimately terminated, as discussed above. In an alternative embodiment, the process is performed on a continuous or semi-continuous basis. In the continuous and semi-continuous process, an additional vessel or plurality of vessels may be utilized to continuously strip away the product stream for cooling and subsequent phase separation.

### Second Reaction Stage

Regardless of how the initial reaction between IBA and HBr are conducted in reactor 100, residual IBA and HBr will be contained in the cooled aqueous stream. To maximize IBB formation, it may be desirable to further react the residual IBA and HBr contained in the cooled aqueous stream in a second reaction stage to form additional IBB (and water). As shown in FIGS. 1 and 4, the second reaction stage preferably occurs in a second (separate) reactor, although in other embodiments, it may occur in the same reactor in which the first reaction occurred. In one embodiment, the reactive distillation unit is a single stage distillation or flash separation as shown in FIG. 1, in order to minimize capital equipment.

As shown in FIG. 1, cooled aqueous stream 8 from reactor 100 is further processed in a second reactor 120 to form additional IBB and maximize overall conversion. Specifically, cooled aqueous stream 8, which comprises unreacted IBA, water, IBB, HBr and possibly TBB is directed to second reactor 120, which preferably is a reactive distillation unit. The HBr and water contained in cooled aqueous stream 8 form an azeotrope, which has a boiling point of about 126° C. In a preferred embodiment, cooled aqueous stream 8 (comprising water, HBr, IBA and IBB) is heated in second reactor 120, preferably to a temperature below the boiling point of the HBr/water azeotrope. In some exemplary embodiments, the cooled aqueous stream 8 is heated in second reactor 120 to a temperature from 30° C. to 120° C., e.g., from 30° C. to 103° C., or to a temperature less than about 126° C., less than about 120° C., less than about 115° C., or less than about 110° C. As shown, second reactor 120 is heated with a heating medium 40, which is pumped around second reactor 120. The HBr and residual IBA react in second reactor 120 when heated to form additional IBB and water. By limiting the reaction temperature to below the HBr/water azeotrope boiling point, the HBr remains substantially in the liquid phase to react with residual IBA. The reaction temperature in second reactor 120 preferably is greater than 91° C., which is the boiling point of IBB. IBB has a boiling point of 91° C., IBA has a boiling point of about 108° C., and the HBr/water azeotrope boils at about 126° C. As a result, IBB that is formed is preferentially liberated to the gas phase and separated from the HBr, IBA and water that remains substantially in the liquid phase. This additional IBB desirably contributes to improved overall IBA conversion. The resultant gaseous mixture, which exits second reactor 120 via supplemental IBB stream 9, may comprise minor amounts of TBB, SBB, NBB, IBA and water in addition to product IBB, and post-processing to remove these components may be desired, as discussed in greater detail below.

Additional HBr, e.g., anhydrous HBr, optionally is added to second reactor 120 to react with the residual IBA, although the excess HBr provided to the first reaction process should render the addition of fresh HBr unnecessary. In addition, catalyst (as described above with respect to the first reaction stage) optionally is added to second reactor 120 during the second reaction stage. In one embodiment, catalyst is employed in the first reaction and is carried over to the second reaction. Additionally or alternatively, fresh catalyst may be added to second reactor 120.

Reverting to FIG. 1, during the reactive distillation and/or after the reactive distillation is substantially completed, residual liquid material from second reactor 120, which typically includes water and HBr is drained from second reactor 120 and forms waste stream 10. In one embodiment, waste stream 10 is further processed, e.g., neutralized, by mixing with base, e.g., soda ash (Na₂CO₃).

As shown, supplemental IBB stream 9 is directed to heat exchanger 122, which cools and condenses stream 9 to form liquid supplemental IBB stream 11, which is directed to reactor 100 for further processing, for example, washing and drying, as discussed below. Ideally, IBB from vessel 140 is combined in reactor 100 with the supplemental IBB from liquid supplemental IBB stream 11, and the IBB from both reaction stages are post-processed together. Of course, in other embodiments, the washing and drying may occur outside of reactor 100, e.g., in separate washing and drying units.

In another embodiment (not shown), the second reaction stage occurs in the same reactor as the first reaction stage, e.g., reactor 100. For example, after the cooled organic stream is removed from reactor 100, the separated cooled aqueous stream may be reintroduced to reactor 100 for further reacting in the second reaction stage. In this embodiment, residual unreacted IBA and HBr that are contained in the cooled aqueous stream are again reacted at an elevated temperature to form additional IBB and water, but in reactor 100 rather than in reactor 120. In one embodiment, the reaction conditions for the second reaction stage are similar to the reaction conditions for the first reaction stage. Alternatively, the second reaction conditions may differ from the reaction conditions of the first reaction. For example, in the second reaction stage, conditions may be modified such that the second reaction stage is conducted as a reactive distillation in reactor 100, in which newly formed IBB is liberated from reactor 100 as a gas for supplemental IBB recovery. As with the embodiment shown in FIG. 1, additional HBr, e.g., anhydrous HBr, may or may not be supplied to reactor 100 during the second reaction stage.

### IBB Post-Processing

As indicated above, the IBB formed in the first and second reaction stages may comprise a minor amount of impurities such as water and IBA, which ideally are substantially removed from the IBB to form a final IBB composition. In a preferred embodiment, the IBB-containing effluent from the first and second reaction stages are combined and post-processed, e.g., washed and dried, together. Of course, in other embodiments, the IBB-containing effluent from the first and second stages may be post-processes separately and optionally combined to form the final IBB composition.

As described in greater detail below, the post-processing may be performed in the first reactor 100 (See FIG. 1) or similar vessel or in a substantially continuous post-processing system (See FIG. 4).

A. Post-Processing in First Reactor

In one embodiment, IBB formed in the first and/or second reaction stages may be processed in reactor 100 to remove impurities, e.g., IBA, water and HBr. Preferably, IBB in vessel 140 (formed in the first reaction stage) is combined with IBB formed in the second reaction stage for combined post-processing. As shown in FIG. 1, IBB from vessel 140 may be directed to reactor 100 via stream 141, and IBB from second reactor 120 may be directed to reactor 100 via stream 11. In another embodiment, the IBB formed in the first reaction stage is post treated while in reactor 100 prior to transfer to a storage vessel such as 140. This eliminates the need of costly materials of construction for the storage vessel. In the embodiment where the second reaction stage occurs in the same reactor as the first reaction stage, the aqueous phase from the second reaction stage is removed from the reactor 100 after completion of the second reaction stage, and the IBB from vessel 140 is added to the IBB in the reactor 100 that was formed in the second reaction stage.

The post-processing preferably includes one or more washing steps in which the IBB composition contained in reactor 100 (preferably a mixture of IBB formed in the first and second reaction stages) is contacted with water and/or a mixture of water and base (e.g., soda ash) to extract water-soluble contaminants contained in the IBB composition. In one embodiment, mixer 102 is used to facilitate the washing step. Alternatively, the washing may be performed in a separate washing unit, as discussed below with reference to FIG. 4. After washing, the resulting aqueous phase is oriented above the washed organic phase, regardless of temperature; no phase inversion is observed. The washed organic phase may be directed to a temporary storage vessel, and the resulting wash medium phase may then be removed from reactor 100 for treatment and disposal. Once the wash medium phase has been removed from reactor 100, the washed organic phase may be reintroduced to reactor 100 for further processing, e.g., additional washing and/or drying.

Residual water removal preferably is performed by adding a drying agent, e.g., molecular sieves, to the washed organic phase contained in reactor 100 or passing the liquid through a bed of the drying agent. In a preferred embodiment, the molecular sieves have pores selected so as to adsorb residual water from the washed organic phase. Additionally or alternatively, the molecular sieve material can be selected so as to remove residual IBA from the washed organic phase. Generally, the molecular sieve material may be selected to remove any impurity that may exist in the washed organic phase. In a preferred embodiment, the molecular sieve material is selected such that both water and IBA are removed from the washed organic phase, thereby forming the final IBB composition. Suitable molecular sieve materials include, for example, mole sieve 3A, 4A and 5A, commercially available from Aldrich.

B. Continuous Post-Processing System

FIG. 4 illustrates a reaction system similar to FIG. 1, but further comprising a substantially continuous post-processing system. In the embodiment shown in FIG. 4, the products of the two reaction stages are post-processed in the same continuous system. Of course, the two streams may be processed separately if desired.

As shown, supplemental IBB stream 9 is cooled in heat exchanger 122 to form liquid supplemental IBB stream 11, which is directed to a washing unit 401. As shown, cooled organic stream 7′ from the first reaction stage is combined with liquid supplemental IBB stream 11 in washing unit 401 to form IBB-containing mixture 410, although in other embodiments the streams may be combined upstream of washing unit 401. In another embodiment, not shown, IBB from the first reaction stage may be temporarily stored in a vessel similar to vessel 140 in FIG. 1 and then directed from vessel 140 to washing unit 401 or combined with liquid supplemental IBB stream 11 upstream of washing unit 401. In still another embodiment, the reaction system does not include cooled organic stream 7′, and the combined phases from the first reaction stage 100 are directed to second reactor 120 via a combined stream 8. Thus, in this embodiment, the aqueous and organic phases from the first reaction stage are directed to the second reaction stage together to form a continuous reaction system that does not require periodic cycling of the first reactor 100 in order to separate the organic phase from the aqueous phase.

In washing unit 401, the IBB-containing mixture 410 is washed with washing medium 402, e.g., preferably water with base, optionally with a base such as soda ash (N₂CO₃) in order to maximize equipment lifetime. In washing unit 401, washing medium 402 preferably contacts the IBB under conditions effective to separate impurities that are soluble in the washing medium from IBB-containing mixture 410. Preferably, for example, IBA and HBr are soluble in the washing medium and IBB is insoluble in the washing medium. Ultimately, washed stream 403 exits washing unit 401 and is directed to a phase separation unit 404. In phase separation unit 404, the aqueous phase is separated from wet IBB phase to form aqueous stream 405 and wet IBB stream 406, which is directed to drying unit 407.

Drying unit 407 preferably utilizes molecular sieves, optionally in a molecular sieve bed 408, having pore characteristics suitable for adsorbing water. In addition, molecular sieves can be selected so as to remove residual IBA from wet IBB stream 406. Suitable molecular sieve materials include, for example, mole sieve 3A, 4A and 5A, commercially available from Aldrich. In a preferred embodiment, the molecular sieves are selected such that both water and IBA are removed from wet IBB phase 406, preferably simultaneously. In a preferred embodiment, drying unit 190 removes at least 50 wt. %, e.g., at least 70 wt. %, or at least 90 wt. % of the water from wet IBB phase 406. In addition, drying unit 190 preferably removes at least 30 wt. %, e.g., at least 50 wt. %, at least 80 wt. % or at least 90 wt. %, of the IBA from wet IBB stream 406. Ultimately, final IBB composition 409 exits drying unit 407 and is directed to a vessel for storage.

### Final IBB Composition

In a preferred embodiment, after washing and drying, the final IBB composition has an alkyl halide, e.g., IBB, concentration of greater than 90%, e.g., greater than 95%, greater than 99% or greater than 99.5%. The overall composition of the final IBB composition preferably is as indicated below in Table 2. In this context, by “overall” it is meant IBB product formed in the first and second reaction stages in combination.

TABLE 2
Exemplary Compositions of Final IBB Composition

| Component | Final IBB Composition (Wt %) | | |
|---|---|---|---|
| Water | <1000 wppm | <500 wppm | <100 wppm |
| HBr | <1000 wppm | <500 wppm | <100 wppm |
| IBA | <3 | <1 | <0.8 |
| IBB | 93-99.5 | 95-99.5 | 97-99.5 |
| SBB | <0.7 | <0.4 | <0.3 |
| TBB | <0.5 | <0.3 | <0.2 |

As indicated above, the processes of the present invention provide the ability to form IBB in surprisingly high yields, without the use of a true distillation step. Embodiments of the invention will become more evident in view of the following non-limiting examples.

### EXAMPLE 1

### First Reaction Stage

A 20 gallon glass-lined steel reactor equipped with an agitator and pumparound loop similar to that shown in FIG. 1 was loaded with 80.6 lbs. (36.6 kg) IBA. Anhydrous HBr was fed via a conduit in the pumparound loop. The reactor was maintained at approximately 75° C. The HBr fed was approximately 123.0 lb (55.8 kg) over 6.5 hours. The reactor was cooled to about 30° C. and the agitator was stopped to allow phase separation. The bottom aqueous phase was removed and collected in a 20 gallon (76 liter) glass lined steel reactor for further reaction. Similar organic phase analysis for various temperatures were found to contain IBB and IBA in the concentrations shown in Table 3. The IBB concentration in the organic phase unexpectedly increased with decreasing temperature.

TABLE 3
Composition of Organic Phase of First Reaction Stage

| | 20° C. | 25° C. | 40° C. |
|---|---|---|---|
| IBB (wt. %) | 95.4 | 92.1 | 88.8 |
| IBA (wt. %) | 2.5 | 4.0 | 6.6 |

### EXAMPLE 2

### Post-Processing of First Stage Product

37.9 lb (17.6 kg) of 10 wt. % soda ash in water was added to the organic phase formed in Example 1 (in the reactor) and mixed. The agitator was turned off and the mixture allowed to phase separate. The bottom organic phase was removed and analyzed. The organic phase contained 95.6 wt. % IBB, 2.85 wt. % TBB, 1.09 wt. % IBA, 0.67 wt. % SBB, based on the total weight of the organic phase. The organic phase was pumped through a mole sieve bed and collected. The resulting dry product was found to contain 95.83 wt. % IBB, 2.85 wt. % TBB, 0.91 wt. % IBA and 0.08 wt. % SBB.

### EXAMPLE 3

### Second Reaction Stage and Post-Processing

The combined HBr rich aqueous phases from two first reaction stage runs as described in Example 1 were collected in a second 20 gallon jacketed glass lined steel reactor (second stage reactor). Hot oil was used as the heating medium to heat the second stage reactor in a manner similar to reactor 120 in FIG. 1. The vent line went to a water cooled condenser similar to 122 in FIG. 1 which emptied back to the first stage reactor 100. After heating to approximately 75° C. in the second stage reactor, the organic contents of the reactor was determined to have 86.57 wt. % IBA, 11.47 wt. % IBB, and 0.89 wt. % TBB. The second stage reactor was gradually heated to 103° C. The overhead product was collected in the first stage reactor. 33.4 lb (15.1 kg) soda ash solution was used to wash the reactor contents. After phase separation, the bottom IBB product phase was transferred to a collection vessel similar to 140 in FIG. 1. The product was pumped through a mole sieve bed. The recovered second reaction stage product contained 82.41 wt. % IBB, 9.89 wt. % TBB, 6.12 wt. % IBA and 0.57 wt. % SBB. The contents remaining in the second stage reactor (ultimately exiting reactor 120 as stream 10) contained 42.4 wt. % HBr and had a density of 1.4 g/ml.

### EXAMPLE 4

### Second Run

The overall process was repeated as described in FIG. 1. The first reaction stage was run at about 80° C. and the second stage at 120° C. The resulting final IBB product, after simple washing and drying through a mole sieve dryer bed, comprised 97.45 wt. % IBB, 0.88 wt. % IBA, 0.26 wt. % SBB, and 0.16 wt. % TBB determined by gas chromatography, and contained 85 wppm water.

Any feature described or claimed with respect to any disclosed implementation may be combined in any combination with any one or more other feature(s) described or claimed with respect to any other disclosed implementation or implementations, to the extent that the features are not necessarily technically incompatible, and all such combinations are within the scope of the present invention. Furthermore, the claims appended below set forth some non-limiting combinations of features within the scope of the invention, but also contemplated as being within the scope of the invention are all possible combinations of the subject matter of any two or more of the claims, in any possible combination, provided that the combination is not necessarily technically incompatible.

What is claimed is:

1. A process for producing an alkyl halide, comprising:
(a) contacting an alcohol with a hydrogen halide in a reactor at elevated temperature under conditions effective to form an initial product mixture comprising the alkyl halide, the alcohol, the hydrogen halide and water;
(b) cooling the initial product mixture to form a cooled organic phase positioned above a cooled aqueous phase; and
(c) separating the cooled organic phase from the cooled aqueous phase.

2. The process of claim 1, further comprising:
(d) heating at least a portion of the cooled aqueous phase under conditions effective to form additional alkyl halide.

3. The process of claim 2, wherein the alcohol is isobutanol, the alkyl halide is isobutyl bromide, and the hydrogen halide is hydrogen bromide.

4. The process of claim 3, wherein anhydrous hydrogen bromide is added to the reactor during the contacting step.

5. The process of claim 4, wherein the cooled organic phase comprises isobutyl bromide in an amount from 95 to 99 wt. %, based on the total weight of the cooled organic phase.

6. The process of claim 4, wherein the elevated temperature is from 70 to 80° C.

7. The process of claim 4, wherein the cooled aqueous phase comprises hydrogen bromide in an amount from 40-50 wt. %, based on the total weight of the cooled aqueous phase.

8. The process of claim 4, wherein the hydrogen bromide and isobutanol are added to the reactor at a molar ratio greater than 1:1.

9. The process of claim 4, wherein the cooling comprises applying an ice bath to the initial product mixture.

10. The process of claim 4, wherein the cooling comprises applying a cooling jacket to the reactor, applying a cooling coil to the reactor, or allowing the initial product mixture to cool to room temperature.

11. The process of claim 4, wherein the hydrogen bromide is added to the reactor at ambient pressure.

12. The process of claim 4, wherein the contacting is performed at ambient pressure.

13. The process of claim 4, wherein the hydrogen bromide is introduced into the reactor by bubbling in the reactor.

14. The process of claim 3, wherein the reactor includes a circulation loop in which at least a portion of the contacting occurs, and wherein a circulation stream comprising the isobutanol, the hydrogen bromide, the isobutyl bromide and the water is pumped through the circulation loop.

15. The process of claim 14, wherein gaseous anhydrous hydrogen bromide is added to the circulation stream.

16. The process of claim 14, wherein the cooling comprises cooling the circulation stream.

17. The process of claim 16, wherein the circulation stream is cooled with a heat exchanger.

18. The process of claim 3, wherein step (d) comprises reactively distilling the cooled aqueous phase.

19. The process of claim 18, wherein a catalyst is added during the reactive distilling step.

20. The process of claim 18, wherein additional HBr is added during the reactive distilling step.

21. The process of claim 18, wherein the hydrogen bromide and the water in the cooled aqueous phase form an azeotrope.

22. The process of claim 21, wherein the reactively distilling comprises heating the azeotrope to a temperature less than the boiling point of the azeotrope.

23. The process of claim 21, wherein the reactively distilling comprises heating the azeotrope to a temperature of less than about 126° C.

24. The process of claim 18, wherein the reactively distilling produces an overhead stream comprising at least a portion of the additional isobutyl bromide.

25. The process of claim 24, wherein the overhead stream is cooled and condensed to form a liquid product stream.

26. The process of claim 3, further comprising washing the isobutyl bromide with water to formed washed isobutyl bromide.

27. The process of claim 26, further comprising drying the washed isobutyl bromide with a molecular sieve material.

28. The process of claim 3, wherein the additional isobutyl bromide is combined with the isobutyl bromide in the cooled organic phase to form a combined crude product stream.

29. The process of claim 28, wherein the combined crude product stream is washed with water and dried to form a final isobutyl bromide product.

30. The process of claim 29, wherein the final isobutyl bromide product comprises at least 95 weight percent isobutyl bromide, based on the total weight of the final isobutyl bromide product.

31. A process for producing isobutyl bromide, comprising:
(a) reacting isobutanol with hydrogen bromide in a reactor to form an aqueous phase situated on top of an organic phase;
(b) cooling the aqueous phase and the organic phase such that the phases invert; and
(c) separating the organic phase from the aqueous phase.

32. The process of claim 31, further comprising:
(d) heating the aqueous phase to form additional isobutyl bromide.

33. The process of claim 32, wherein anhydrous hydrogen bromide is added to the reactor during the reacting step.

34. The process of claim 33, wherein the organic phase after cooling comprises isobutyl bromide in an amount from 95 to 99 wt. %, based on the total weight of the organic phase.

35. The process of claim 33, further comprising
(e) pumping a circulation stream comprising the isobutanol, the hydrogen bromide, the isobutyl bromide and the water to the reactor in a circulation loop.

36. The process of claim 35, wherein gaseous anhydrous hydrogen bromide is added to the circulation stream.

37. The process of claim 35, wherein the cooling comprises cooling the circulation stream.

38. The process of claim 32, wherein step (d) comprises reactively distilling the aqueous phase.

39. The process of claim 38, further comprising washing the isobutyl bromide with water to form washed isobutyl bromide.

40. The process of claim 39, further comprising drying the washed isobutyl bromide with a molecular sieve material.

41. The process of claim 38, wherein the reactively distilling produces an overhead stream comprising at least a portion of the additional isobutyl bromide.

42. The process of claim 41, wherein the overhead stream is cooled and condensed to form a liquid product stream.

43. The process of claim 32, wherein the additional isobutyl bromide is combined with the isobutyl bromide in the organic phase to form a combined crude product stream.

44. The process of claim 43, wherein the combined crude product stream is washed with water and dried to form a final isobutyl bromide product.

45. The process of claim 44, wherein the final isobutyl bromide product comprises at least 95 weight percent isobutyl bromide, based on the total weight of the final isobutyl bromide product.

46. A reaction system for forming an alkyl halide, comprising:
(a) a reactor for contacting a hydrogen halide with an alcohol under conditions effective to form an organic phase comprising the alkyl halide and an aqueous phase comprising water, the hydrogen halide and the alcohol; and
(b) means for cooling and separating the organic phase and aqueous phase to form a cooled organic phase and a cooled aqueous phase.

47. The reaction system of claim 46, further comprising:
(c) a reactive distillation unit in fluid communication with the reactor for receiving at least a portion of the cooled aqueous phase and configured to form additional alkyl halide from the hydrogen halide and the alcohol contained in the cooled aqueous phase.

48. The reaction system of claim 47, in which the cooled organic phase is directed to a washing vessel and an overhead stream from the reactive distillation unit directs the additional alkyl halide to the washing vessel.

49. The reaction system of claim 48, wherein the alcohol is isobutanol, the alkyl halide is isobutyl bromide, and the hydrogen halide is hydrogen bromide.

50. The reaction system of claim 49, wherein the reactor is the wash vessel.

51. The reaction system of claim 49, wherein the wash vessel is in fluid communication with a drying unit.

52. The reaction system of claim 49, wherein the reaction system is a semi-continuous reaction system.

53. The reaction system of claim 49, wherein the reaction system is a batch reaction system.

54. A continuous reaction system for forming an alkyl halide, comprising:
(a) a reactor for contacting a hydrogen halide with an alcohol under conditions effective to form a product mixture comprising the alkyl halide, water, the hydrogen halide and the alcohol; and
(b) a reactive distillation unit in fluid communication with the reactor for receiving at least a portion of the product mixture and configured to form a crude alkyl halide product mixture comprising alkyl halide from the reactor and additional alkyl halide formed from the hydrogen halide and the alcohol contained in the product mixture.

55. The continuous reaction system of claim 54, wherein the alcohol is isobutanol, the alkyl halide is isobutyl bromide, and the hydrogen halide is hydrogen bromide.

56. The continuous reaction system of claim 55, wherein the reactive distillation unit is in fluid communication with a washing unit, and the crude alkyl halide product mixture is washed in the washing vessel to form a washed stream.

57. The continuous reaction system of claim 56, wherein the washing unit is in fluid communication with a phase separation unit for separating an organic phase comprising the alkyl halide, and an aqueous phase.

58. The continuous reaction system of claim 57, wherein the phase separation unit is in fluid communication with a drying unit configured to dry the organic phase to form a final alkyl halide product.
